(19) United States
(12) Patent Application Publication
(10) Pub. No.: US 2022/0016294 A1
(43) Pub. Date: Jan. 20, 2022

(54) MANAGEMENT APPARATUS, MANAGEMENT SYSTEM, INFORMATION GENERATION METHOD, AND PROGRAM FOR MANAGING RECORD OF PROCESS

(71) Applicant: Canon Kabushiki Kaisha, Tokyo (JP)

(72) Inventors: Motohiro Furusawa, Shizuoka (JP); Ichiro Yasumaru, Shizuoka (JP)

(21) Appl. No.: 17/290,278

(22) PCT Filed: Nov. 14, 2019
(86) PCT No.: PCT/JP2019/044755

§ 371 Date: Apr. 30, 2021

(30) Foreign Application Priority Data
Nov. 28, 2018 (JP) 2018-222690
Jul. 16, 2019 (JP) 2019-131400

(51) Int. Cl. A61L 2/28; A61L 2/24; G16H 40/40

(52) U.S. Cl. CPC A61L 2/28; A61L 2/24; A61L 2202/24; A61L 2202/14; G16H 40/40

(57) ABSTRACT

A management apparatus that manages records of a predetermined process carried out sing test paper, the test paper having a discoloration area that changes color in accordance with an achievement degree of the predetermined process, is presented. The management apparatus includes: holding means for holding, for each predetermined process, a record in which information that, indicates a date/time at which the predetermined process was carried out and a determination result of the predetermined process determined based on the color of the discoloration area of the test paper is recorded; and generating means for generating information for determining transition of quality of the predetermined process based on the information recorded in the records.

### TECHNICAL FIELD

The present invention relates to a technique for utilizing records of a predetermined process.

### BACKGROUND ART

Sterilization targets, such as medical tools, are subjected to sterilization processes at hospitals. At this time, what is known as a chemical indicator (“CI” hereinafter) for sterilization is used in order to determine the achievement degree of the sterilization process on the sterilization targets. A CI has a discoloration area that changes color in accordance with the achievement degree of the conditions required for the sterilization process, which uses a sterilizing agent (vapor, hydrogen peroxide, or the like). Japanese Patent No. 3414291 discloses a management apparatus that manages sterilization process records. According to Japanese Patent No. 3414291, the management apparatus includes CI determination results in the sterilization process records.

Also, medical tools are washed after use at hospitals. At this time, a washing indicator is used in order to determine the achievement degree of the washing process. A washing indicator has a discoloration area that changes color in accordance with the achievement degree of the conditions required for the washing process, which uses a washing agent.

In recent years, emphasis has been placed on keeping the quality of sterilization and washing processes high. Although Japanese Patent No. 3414291 discloses including CI determination results in sterilization process records, it does not disclose a method for improving the quality of the sterilization process based on the sterilization process records.

### SUMMARY OF INVENTION

According to an aspect of the present invention, a management apparatus that manages records of a predetermined process carried out using test paper, the test paper having a discoloration area that changes color in accordance with an achievement degree of the predetermined process, is presented. The management apparatus includes: holding means for holding, for each predetermined process, a record in which information that indicates a date/time at which the predetermined process was carried out and a determination result of the predetermined process determined based on the color of the discoloration area of the test paper is recorded; and generating means for generating information for determining transition of quality of the predetermined process based on the information recorded in the records.

Further features of the present invention will become apparent from the following description of exemplary embodiments with reference to the attached drawings.

### BRIEF DESCRIPTION OF DRAWINGS

FIG. 1 is a diagram illustrating the configuration of a management system including a management apparatus according to one embodiment.

FIG. 2 is a diagram illustrating a CI according to one embodiment.

FIG. 3 is a diagram illustrating the configuration of a determination apparatus according to one embodiment.

FIGS. 4A and 4B are diagrams illustrating history tables according to one embodiment.

FIG. 5 is a diagram illustrating information provided by a management apparatus according to one embodiment.

FIG. 6 is a diagram illustrating information provided by a management apparatus according to one embodiment.

FIG. 7 is a diagram illustrating information provided by a management apparatus according to one embodiment.

FIGS. 8A and SB are diagrams illustrating history tables according to one embodiment.

FIG. 9 is a diagram illustrating information provided by a management apparatus according to one embodiment.

FIG. 10 is a diagram illustrating formation provided by a management apparatus according to one embodiment.

FIG. 11 is a diagram illustrating information provided by a management apparatus according to one embodiment.

FIG. 12 is a diagram illustrating a history table according to one embodiment.

FIG. 13 is a diagram illustrating information provided by a management apparatus according to one embodiment.

FIG. 14 is a diagram illustrating information provided by a management apparatus according to one embodiment.

FIG. 15 is a diagram illustrating one example of a physical indicator according to one embodiment.

FIG. 16 is a diagram illustrating a history table according to one embodiment.

FIG. 17 is a function block diagram of a management apparatus according to one embodiment.

### DESCRIPTION OF EMBODIMENTS

Hereinafter, exemplary embodiments of the present invention will be described with reference to the drawings. Note that the following embodiments are to be taken as examples only, and the present invention is not intended to be limited by the embodiments. In each drawing described below, constitutional elements that are unnecessary to describe the embodiments are omitted. Although the following describes the embodiments based on a sterilization process, the present invention can also be applied to a washing process. If the present invention is applied to a washing process, a sterilization process, a sterilization target, a CI, and a sterilization processing apparatus referred to below should be taken as a washing process, a washing target, a washing indicator, and a washing processing apparatus, respectively.

### First Embodiment

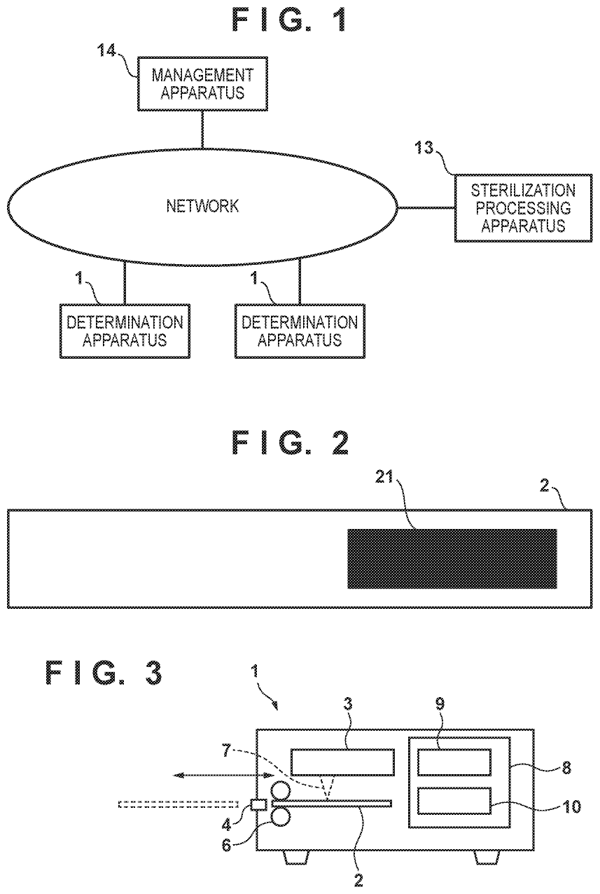

FIG. 1 illustrates a management system including a sterilization process management apparatus 14 according to the present embodiment. The management system includes one or more determination apparatuses 1, the management apparatus 14, and a sterilization processing apparatus 13 that are connected to each other via a network. Each determination apparatus 1, the sterilization processing apparatus 13, and the management apparatus 14 are capable of communicating with each other via the network.

FIG. 2 illustrates a CI 2 according to the present embodiment. The CI 2 is a sheet-shaped test paper, and has on its surface a discoloration area 21 which has been subjected to a chemical process. The discoloration area 21 changes color in accordance with the achievement degree of a sterilization process. In the present embodiment, the discoloration area 21 of the CI 2 is configured to start with a color value C#1 in an initial state, change to color values C#2 to C#9 in accordance with the achievement degree of the sterilization process, and ultimately stabilize at a color value C#10. In the following description, the achievement degree of the sterilization process when the discoloration area 21 has a color value C#k (k is an integer from 1 to 10) will be referred to as an achievement degree #k. Note that the discoloration area 21 continuously changes from the color value C#1 to the color value C#10, rather than having one of the color values C#1 to C#10. That is, the discoloration area 21 may have a color value between a color value C#m (m is an integer from 1 to 9) and a color value C#m+1. In the present embodiment, the achievement degree of the sterilization process is evaluated using nine levels. Specifically, when the color of the discoloration area 21 is within a range between the color value C#m and the color value C#m+1, the level of the sterilization process is determined as Level #m. For example, Levels #8 and #9 are OK levels, and Levels #1 to #7 are NG (NO GOOD) levels. The level of the sterilization process being Level #m means that the achievement degree of the sterilization process is within a range between an achievement degree #m and an achievement degree #m+1.

FIG. 3 is a cross-sectional view of the determination apparatus 1. The determination apparatus 1 includes an insertion portion 4 for a CI 2, and a conveyance roller pair 6 that conveys the CI 2, which has been inserted into the insertion portion 4, to a measurement position on the downstream side. When an operator inserts the CI 2 into the insertion portion 4 as indicated by the dotted line in FIG. 3, the conveyance roller pair 6 conveys the CI 2 to the measurement position, which is indicated by the solid line. A measurement unit 3 is arranged on the side opposite from the measurement position. The measurement unit 3 uses light 7 to read an optical characteristic value pertaining to the color of the discoloration area 21 on the CI 2. For example, the measurement unit 3 is a spectrometer and measures the spectral reflectivity of the discoloration area 21. After the CI 2 is measured by the measurement unit 3, the CI 2 is discharged from the determination apparatus 1 by rotating the conveyance roller pair 6 in reverse.

A control unit 8 controls the determination apparatus 1. Various types of information used for measurement are stored in a storage unit 9 of the control unit 8. A computation processing unit 10 of the control unit 8 determines the color value of the discoloration area 21 of the CI 2 based on the result of the measurement by the measurement unit 3. For example, if the measurement unit 3 measures the spectral reflectivity of the discoloration area 21, the computation processing unit 10 determines the color value of the discoloration area 21 based on the spectral reflectivity of the discoloration area 21. Further, the computation processing unit 10 determines the level of the sterilization process by comparing the determined color value of the discoloration area 21 with the color values C#1 to C#10, Specifically, if the determined color value of the discoloration area 21 is between color values C#m and C#m+1, the level of the sterilization process is determined as Level #m. Further, if the determined level is Level #8 or #9, the computation processing unit 10 determines that the level is “OK”, and otherwise determines that the level is “NG”. In the present embodiment, the level of the sterilization process is evaluated using nine levels, but the level can be evaluated using any number of levels that is two or more. The control unit 8 of the determination apparatus 1 transmits the result of the sterilization process that is determined based on the color of the discoloration area 21 of the CI 2, as a determination result, to the management apparatus 14. The determination result includes either one or both of pieces of information that indicates the determined level of the sterilization process and information that indicates whether the sterilization process is good or bad, in other words, OK or NG.

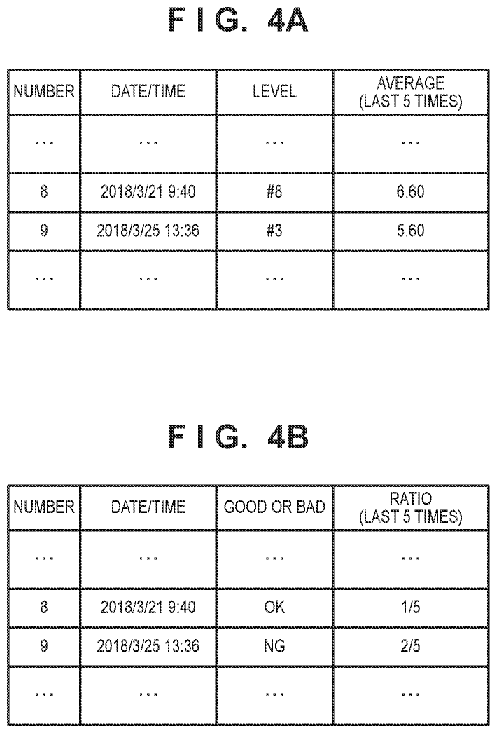

FIG. 4A illustrates one example of a sterilization process history table that is managed by the management apparatus 14. Upon receiving a determination result from the determination apparatus 1, the management apparatus 14 adds a record to the history table, assigns a number to the record, and records the number in a “Number” field. Also, the management apparatus 14 records a date/time that is held by the management apparatus 14 when the determination result is received, in a “Date/Time” field of the added record. Note that a configuration is also possible in which a date/time at which the determination result was determined is included in the determination result that is transmitted from the determination apparatus 1 to the management apparatus 14, and the management apparatus 14 records the date/time included in the determination result in the date/time field. Further, the management apparatus 14 records a level that is included in the received determination result, in a “Level” field of the added record. Further, the management apparatus 14 records an average value of the five latest levels in an “Average” field of the added record. For example, when recording the determination result received from the determination apparatus 1 in the No. 8 record, the management apparatus 14 stores an average value of levels that are recorded in the level fields of Nos. 4 to 8 records, in the average field of the No. 8 record. Thus, the moving average of levels recorded in records that are arranged in chronological order is stored in the average field.

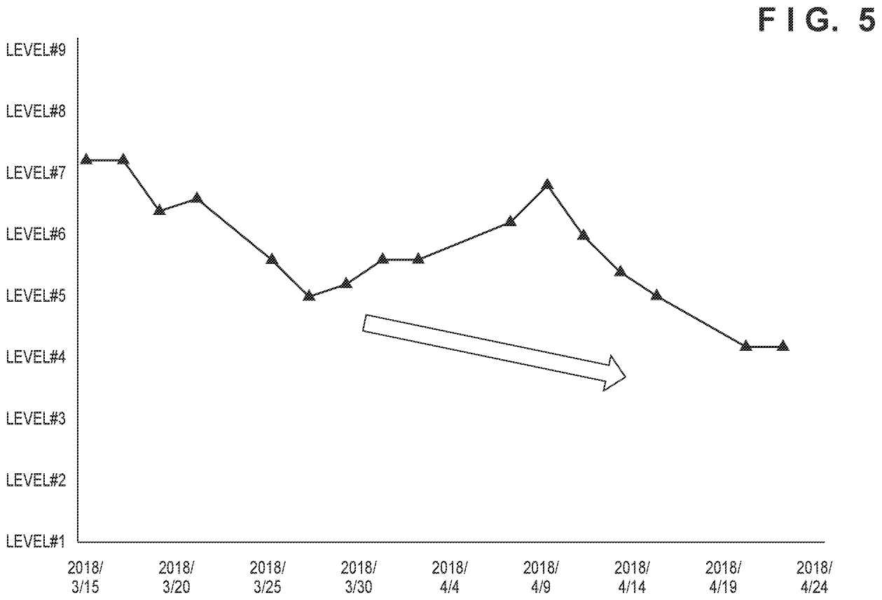

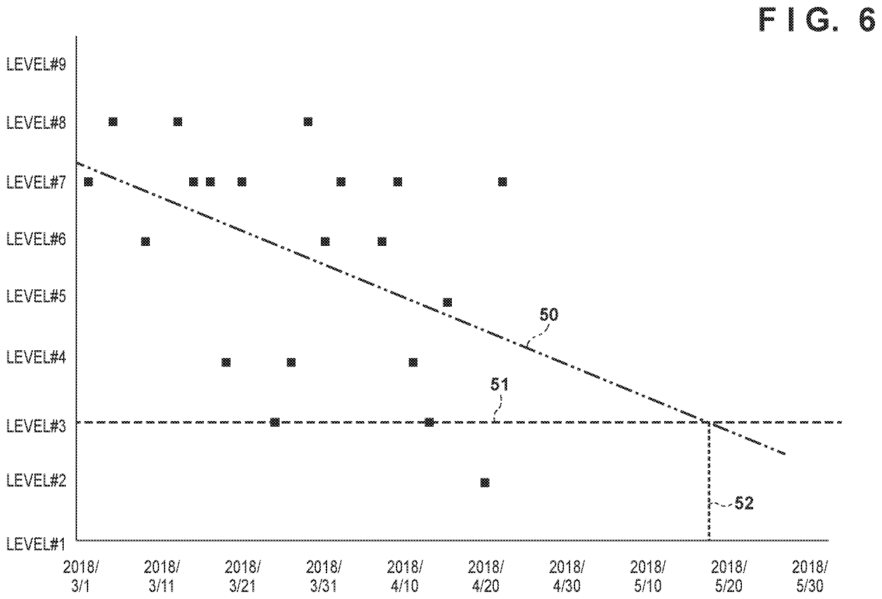

FIG. 5 illustrates one example of a graph that is created based on the history table illustrated in FIG. 4A. In the graph illustrated in FIG. 5, the horizontal axis indicates values in the date/time field, and the vertical axis indicates values in the average field. From the graph in FIG. 5, it can be found that the level of the sterilization process decreases over time. That is, from the graph in FIG. 5, it can be found that the quality of the sterilization process has a tendency to decrease. FIG. 6 illustrates another example of a graph that is created based on the history table illustrated in FIG. 4A. In the graph illustrated in FIG. 6, the horizontal axis indicates values in the date/time field, and the vertical axis indicates values in the level field. Also, each point in the graph in FIG. 6 corresponds to each record in the history table. A straight line 50 in FIG. 6 is obtained by approximating the points in the graph, and can be obtained using various statistical methods, such as the least squares method, for example. That is, the straight line 50 corresponds to a relational expression between the date/time and the level that is obtained based on the points in the graph. In the present embodiment, a relational expression corresponding to a straight line, that is, a linear function is used, but it is possible to use any relational expression that, describes the relationship between the date/time and the level. From the straight line 50 in FIG. 6, it can be found that the level of the sterilization process has a tendency to decrease over time. That is, from the graph in FIG. 6, it can be found that the quality of the sterilization process has a tendency to decrease. Note that it is possible to determine a future level of the sterilization process by extending the straight line 50 from the current point in time to a future point in time, assuming that the current tendency will continue. For example, in FIG. 6, a threshold value is set to Level #3, and a value on the horizontal axis that corresponds to the point of intersection between a straight line 51 that corresponds to the threshold value and the straight line 50 is indicated by a straight line 52. From the graph in FIG, 6, it can be predicted that an average level of the sterilization process will reach Level #3 around the date/time that corresponds to the straight line 52.

FIG. 4B illustrates another example of a sterilization process history table that is managed by the management apparatus 14. Upon receiving a determination result from the determination apparatus 1, the management apparatus 14 adds a record to the history table and records a date/time in the date/time field, as is the case with the history table illustrated in FIG. 4A. Also, the management apparatus 14 records, in a “Good/Bad” field of the added record, good or bad, in other words, OK or NG, that is included in the received determination result. Also, the management apparatus 14 calculates, for each record, the ratio of NG using values recorded in the good/bad fields of a predetermined number of successive records that are arranged in chronological order, which are five successive records in this example, and records the ratio in a “Ratio” field. For example, when recording the determination result in the No. 8 record, the management apparatus 14 stores, in the ratio field, a value that is obtained by dividing the number of records in which NG is recorded in the good/bad field, out of Nos. 4 to 8 records, by five.

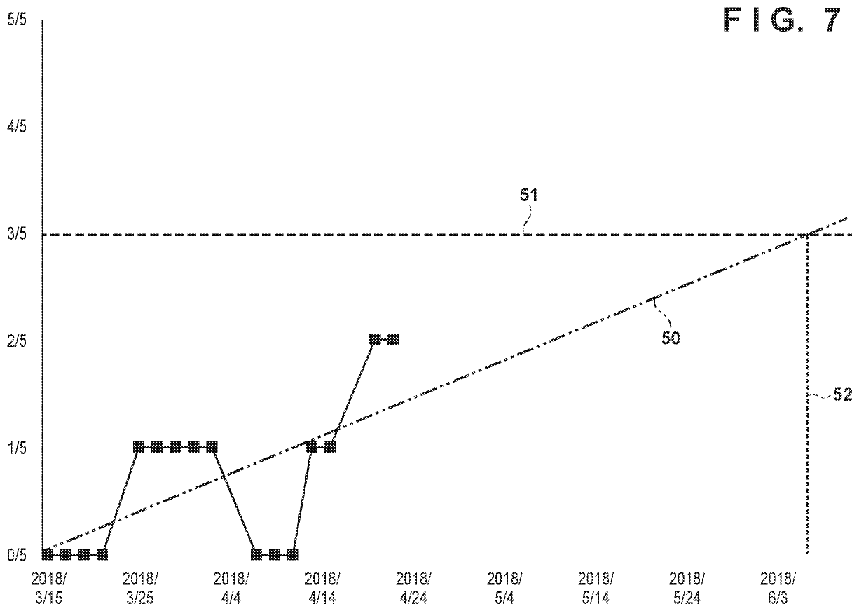

FIG. 7 illustrates one example of a graph that is created based on the history table illustrated in FIG. 4B. In the graph illustrated in FIG. 7, the horizontal axis indicates values in the date/time field, and the vertical axis indicates values in the ratio field. Also, each point in the graph in FIG. 7 corresponds to each record in the history table. A straight line 50 in FIG. 7 is obtained by approximating the points in the graph. From the straight line 50, it can be found that the ratio at which a determination of “NO” is made for the sterilization process increases over time. That is, from the graph in FIG. 7, it can be found that the quality of the sterilization process has a tendency to decrease. In FIG. 7, a threshold value is set to an NG ratio 3/5, and a value on the horizontal axis that corresponds to the point of intersection between a straight line 51 that corresponds to the threshold value and the straight line 50 is indicated by a straight line 52. From the graph in FIG. 7, it can be predicted that the ratio of NG will reach 3/5 when averaged, around the date/time that corresponds to the straight line 52.

If results of the sterilization process are recorded as described above, whether the quality of the sterilization process has a tendency to increase or a tendency to decrease can be determined. Furthermore, it is possible to predict the level of the quality of the sterilization process at a future point in time or predict when the quality will reach a predetermined level. Also, if the above-described tendency is understood, currently performed operations relating to the sterilization process can be improved. Note that, in the present embodiment, the average value of levels (FIG. 4A) and the ratio of NG (FIG. 4B) are respectively calculated as the average and the ratio of the past five records, but this is one example, and the average or the ratio of any other number of records can be used. Also, the ratio of OK can also be used, rather than the ratio of NG.

Note that the sterilization process is performed in a state in which the CI 2 and one or more sterilization targets are packaged in a packaging member, for example. In the following description, one or more sterilization targets that are packaged in one packaging member will be referred to as a set. The number of sets that can be simultaneously sterilized depends on the size of the sterilization processing apparatus 13, and some sterilization processing apparatuses 13 can sterilize only one set at a time, whereas other sterilization processing apparatuses 13 can simultaneously sterilize a plurality of sets. Further, the sterilization process is performed in a state in which, in addition to CIs that are packaged together with sets, a package test pack (PCD) in which a CI is packaged is disposed at a predetermined place inside the sterilization processing apparatus 13 in order to determine the achievement degree of the sterilization process. Each record in the present embodiment corresponds to a record of the sterilization process that is performed on one set, for example. Note that the level of the sterilization process that is performed on one set is determined based on the discoloration area 21 of the CI 2 that is packaged together with the set. Accordingly, if the sterilization process is simultaneously performed on a plurality of sets by one sterilization processing apparatus 13, records of the respective sets are added to the history table. Note that a configuration is also possible in which the sterilization process that is performed by one sterilization processing apparatus 13 is recorded as one record, rather than the sterilization process that is performed on one set. In this case, a level that is determined based on the discoloration area 21 of the CI 2 packaged in the PCD is recorded in the record. That is, in this case, one record is added per sterilization process, irrespective of the number of sets that are sterilized in one sterilization process by one sterilization processing apparatus 13.

### Second Embodiment

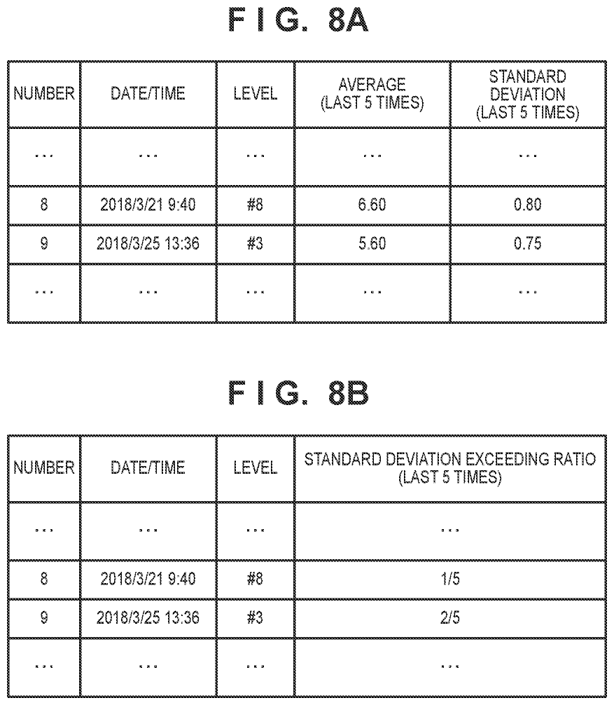

Next, a second embodiment will be described focusing on differences from the first embodiment. In the first embodiment, whether the quality of the sterilization process has a tendency to increase or a tendency to decrease is determined based on the levels of the sterilization process, the average value of levels, or the ratio of NO or OK. In the present embodiment, a standard deviation is taken into consideration. FIG. 8A illustrates one example of a sterilization process history table that is managed by the management apparatus 14 in the present embodiment. Pieces of content recorded in the number field, the date/time field, the level field, and the average field are the same as those recorded in the history table illustrated in FIG. 4A described in the first embodiment. In the present embodiment, the management apparatus 14 records, in a “Standard deviation” field, the standard deviation of the five latest levels recorded in the level fields. In the present embodiment, the value in the standard deviation field of a record is calculated using the five latest records that are used for calculating the value in the average field of the same record. However, it is not necessary that the number of the latest records that are used for calculating the value in the standard deviation field of a record is the same as the number of the latest records that are used for calculating the value in the average field of the same record.

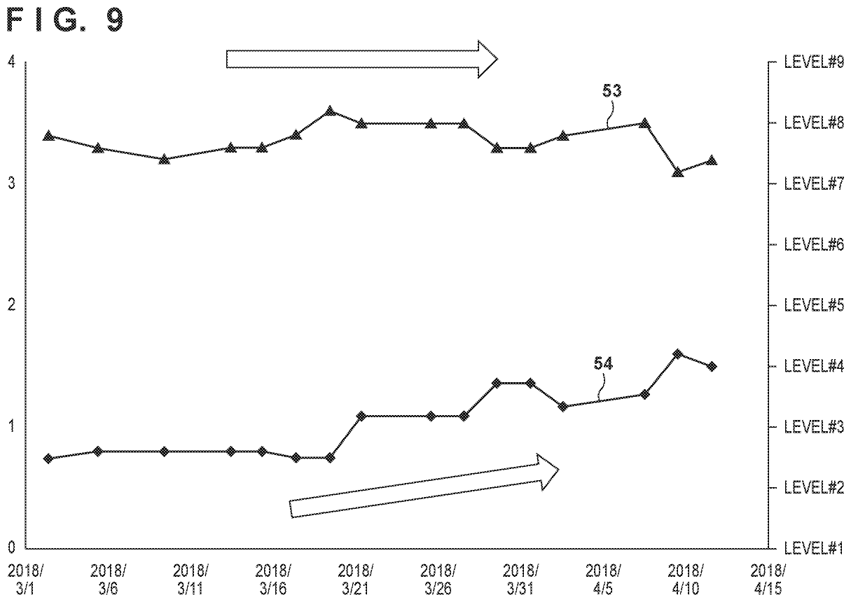

FIG. 9 illustrates one example of graph data that is created based on the history table illustrated in FIG. 8A. A graph line 53 in FIG. 9 indicates values in the average field, similarly to the graph in FIG. 5 in the first embodiment. On the other hand, a graph line 54 indicates values in the standard deviation field. The graph line 53 shows that the level of the sterilization process is kept at substantially the same level overall. On the other hand, the graph line 54 shows that the standard deviation of the level of the sterilization process increases over time. That is, the graph line 54 shows that variation in the quality of the sterilization process increases.

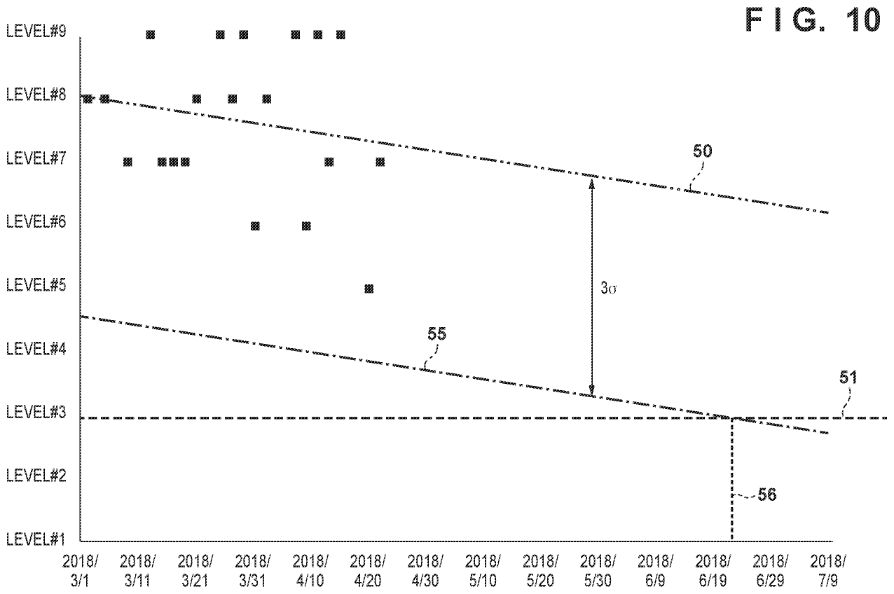

FIG. 10 illustrates another example of a graph that is created based on the history table illustrated in FIG. 8A. In the graph illustrated in FIG. 10, the horizontal axis indicates values in the date/time field, and the vertical axis indicates values in the level field. Each point in the graph in FIG. 10 corresponds to each record in the history table, as is the case with FIG. 6. A straight line 50 in FIG. 10 is obtained by approximating the points in the graph. From the straight line 50, it can be found that the level of the sterilization process has a tendency to decrease. However, there still remains a long period until the average level of the sterilization process reaches Level #3. On the other hand, a straight line 55 is obtained by reducing the level indicated by the straight line 50 by triple (3σ) the standard deviation of levels shown in the level fields of records that are used for calculating the straight line 50. Note that the straight line 55 indicates the probability of the level indicated by the straight line 55 being achieved. In FIG. 10, a value on the horizontal axis that corresponds to the point of intersection between a straight line 51 that corresponds to Level #3 and the straight line 55 is indicated by a straight line 56. From the graph in FIG. 10, it is possible to predict the probability of a determination of “Level #3” being made around the date/time that corresponds to the straight line 56. in the present embodiment, the straight line 55 is obtained by subtracting triple the standard deviation from the level indicated by the straight line 50, but how many times the standard deviation is to be subtracted is determined depending on the probability to be determined.

FIG. 8B illustrates another example of a sterilization process history table that is managed by the management apparatus 14. Pieces of content recorded in the number field, the date/time field, and the level field are the similar to those recorded in the history table illustrated in FIG. 8A. The management apparatus 14 calculates, for each record, an average value and a standard deviation of the level of the sterilization process based on the level fields of a predetermined number of successive records arranged in chronological order, the number of records being five in this example. Subsequently, the management apparatus 14 calculates a difference between a level that is recorded in the level field of a record and the average value that is calculated for the same record, and compares the calculated difference with the standard deviation that is calculated for the same record. Then, the management apparatus 14 determines, for each record, a comparison result that indicates whether the difference is larger than the standard deviation. Finally, the management apparatus 14 stores, in a “Standard deviation exceeding ratio” field, a ratio at which the comparison result indicates that the difference is larger than the standard deviation, based on comparison results determined for the five latest records.

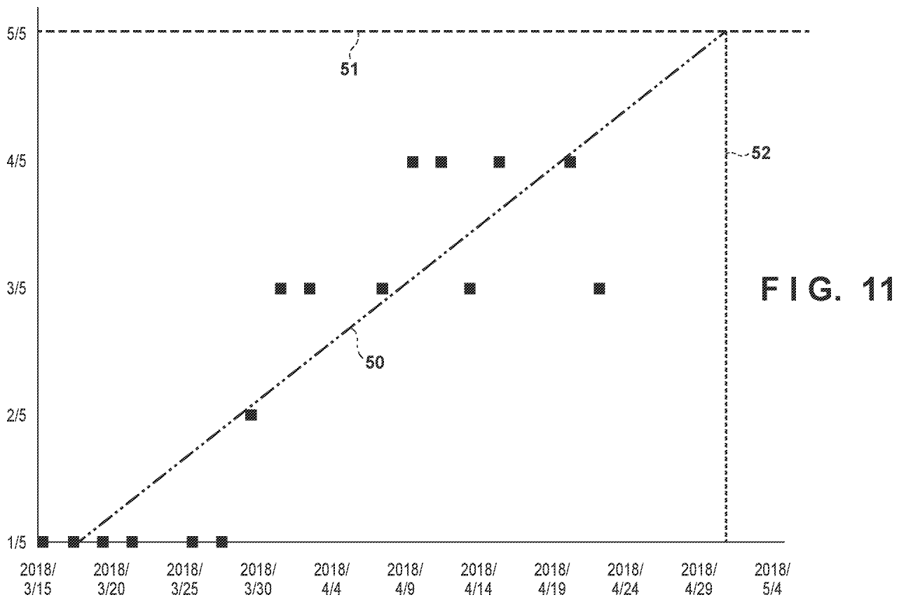

FIG.11 illustrates one example of a graph that is created based on the history table illustrated in FIG. 8B. In the graph illustrated in FIG. 11, the horizontal axis indicates values in the date/time field, and the vertical axis indicates values in the standard deviation exceeding ratio field. Each point in the graph in FIG. 11 corresponds to each record in the history table. A straight line 50 in FIG. 11 is obtained by approximating the points in the graph. From the straight line 50, it can be found that the ratio at which the difference from the average value is larger than the standard deviation increases over time. That is, the graph in FIG. 11 shows that variation in the quality of the sterilization process has a tendency to increase. In FIG. 11, a threshold value is set to a ratio 5/5, and a value on the horizontal axis that corresponds to the point of intersection between a straight line 51 that corresponds to the threshold value and the straight line 50 is indicated by a straight line 52. From the graph in FIG. 11, it can be predicted that the ratio at which the difference from the average value is larger than the standard deviation will reach 5/5 when averaged, around the date/time that corresponds to the straight line 52. In FIG. 11, the difference between the level and the average value is compared with the standard deviation, which serves as a threshold value, but any multiple of the standard deviation can be used as the threshold value. For example, double or triple the standard deviation, that is, an integral multiple of the standard deviation can be used as the threshold value. Further, in FIG. 8B, the ratio at which the difference is larger than the threshold value is recorded in each record, but a configuration is also possible in which the ratio at which the difference is equal to or smaller than the threshold value is recorded in each record.

As described above, in the present embodiment, it is possible to determine the tendency of variation in the quality of the sterilization process, that is, whether variation in the quality has a tendency to increase or a tendency to decrease, in addition to the overall tendency of the quality of the sterilization process described in the first embodiment.

### Third Embodiment

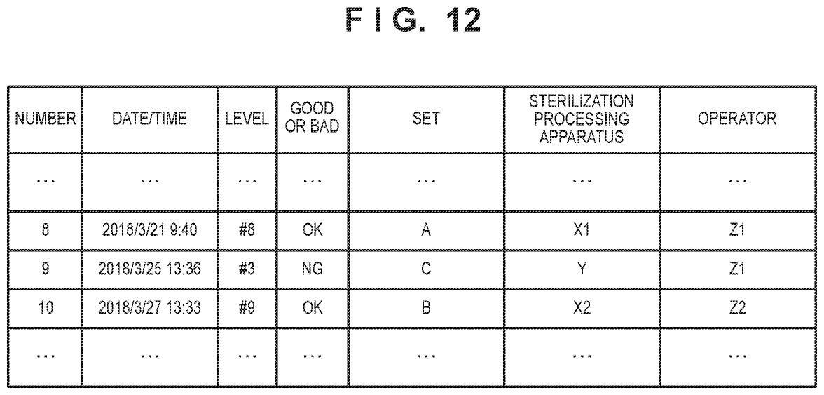

Next, a third embodiment will be described focusing on differences from the first embodiment. FIG. 12 illustrates one example of a sterilization process history table that is managed by the management apparatus 14 in the present embodiment. Pieces of content recorded in the number field, the date/time field, the level field, and the good/bad field are similar to those respectively recorded in the fields of the same names in the first embodiment. Further, pieces of information for identifying the type of set subjected to the sterilization process, information for identifying a sterilization processing apparatus used for the sterilization process, and information for identifying an operator are stored in a “Set” field, a “Sterilization processing apparatus” field, and an “Operator” field, respectively. An operator who has carried out the sterilization process inputs these pieces of information by operating the management apparatus 14. Note that types of sets are determined depending on whether one or more sterilization targets that are packaged in a packaging member are the same as or different from one or more sterilization targets that are packaged in another packaging member. That is, if one or more sterilization targets that are packaged in a packaging member are the same as one or more sterilization target that are packaged in another packaging member, these sets are of the same type. In this example, sets are classified into three types A, B, and C. In this example, two types of sterilization processing apparatuses X and Y are used, and these sterilization processing apparatuses include two sterilization processing apparatuses X, one of which is denoted with X1 and the other is denoted with X2. Note that the sterilization processing apparatuses X can be used for the sterilization process of the sets A and B, and the sterilization processing apparatus Y can be used for the sterilization process of the set C. Further, there are two operators Z1 and Z2.

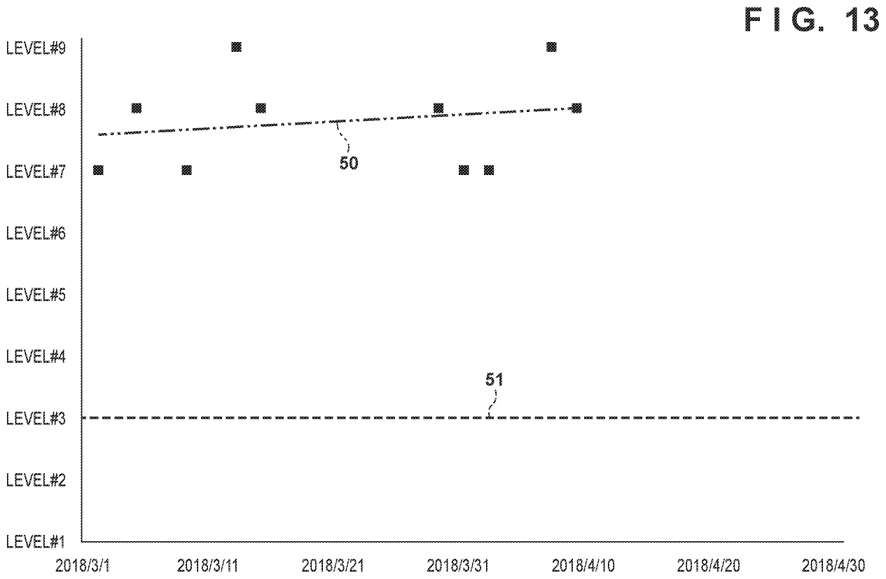

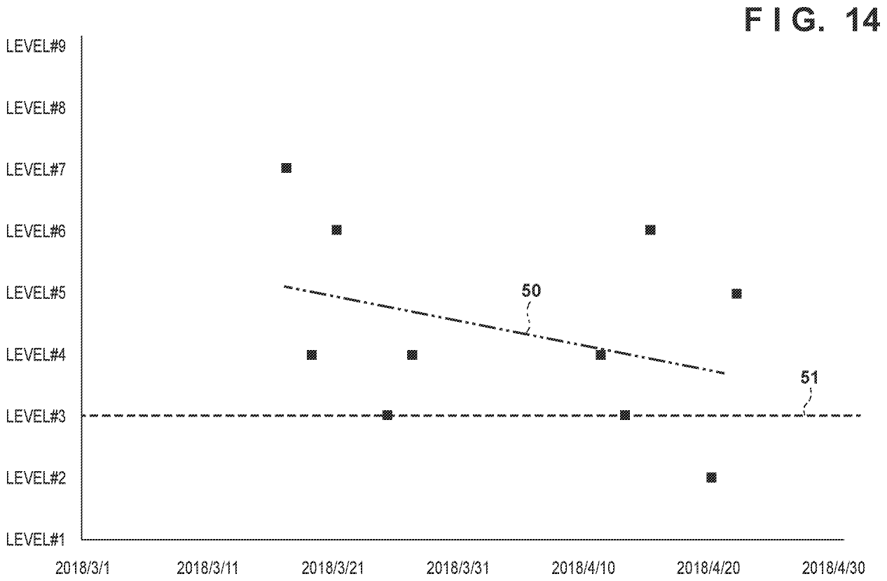

FIGS. 13 and 14 each illustrate one example of a graph that is created based on the history table illustrated in FIG. 12. Note that the graphs in FIGS. 13 and 14 are similar to the graph in FIG. 6. However, the graph in FIG. 13 is only based on records of sterilization processes performed by the operator Z1, and the graph in FIG. 14 is only based on records of sterilization processes performed by the operator Z2. FIG. 13 shows that the sterilization processes performed by the operator Z1 are kept at a high level. On the other hand, FIG. 14 shows that levels of the sterilization processes performed by the operator Z2 are generally lower than levels of the sterilization processes performed by the operator Z1, and have a tendency to decrease over time.

For example, assume that levels of sterilization processes performed using the sterilization processing apparatus Y are particularly low out of the sterilization processes performed by the operator Z2, and levels of sterilization processes performed by the operator Z1 using the sterilization processing apparatus Y do not differ from levels of sterilization processes performed by the operator Z1 using the sterilization processing apparatuses X1 and X2. In such a case, it is possible to suppose that, there is a problem in the operations of the sterilization processing apparatus Y made by the operator Z2 or the manner in which the operator Z2 handles sets C. In the present embodiment, transition of the quality of the sterilization process is determined for each operator, but it is also possible to determine transition of the quality of the sterilization process for each sterilization processing apparatus or each type of set. Alternatively, if a plurality of apparatuses of the same type, such as the sterilization processing apparatuses X1 and X2, are used, transition of the quality of the sterilization process can be determined considering the type of the apparatus as a unit, that is, with respect to all sterilization processes performed using the sterilization processing apparatuses X1 and X2.

As described above, related information that relates to the sterilization process is recorded together with the result of the sterilization process. Note that the related information is the sterilization processing apparatus used for the sterilization process, the set subjected to the sterilization process, the operator, or the like, for example. Thus, it is possible to generate information for determining transition of the quality of the sterilization process, with respect to each operator, each sterilization processing apparatus, each type of sterilization processing apparatus, and each type of set. Based on these pieces of information, it is possible to identify, as a cause of a reduction in the quality of the sterilization process, a specific operator, a specific sterilization processing apparatus, or a specific set, for example, and use the result of the identification for improving the quality of the sterilization process.

### Fourth Embodiment

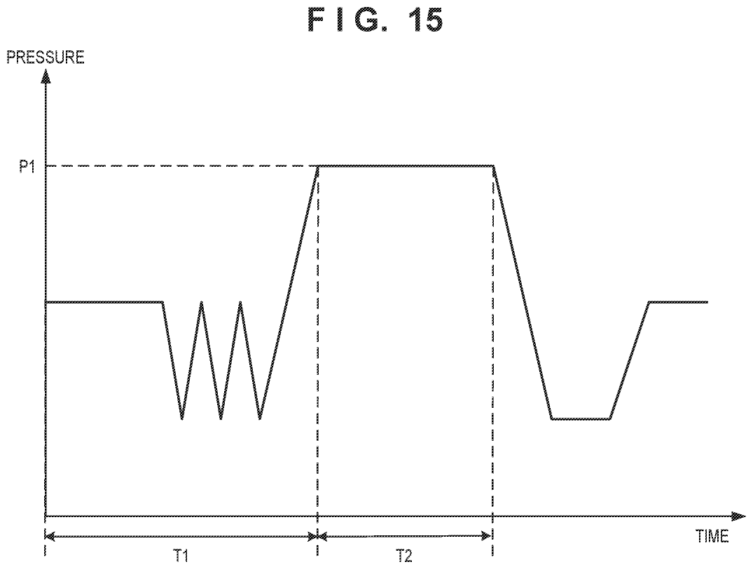

The sterilization processing apparatus 13 records temporal changes in various physical operation parameters (physical amounts), such as temperature and pressure, when performing the sterilization process. Temporal changes in these parameters are also called physical indicators, in contrast to the chemical indicator (CI). FIG. 15 illustrates one example of a physical indicator. FIG. 15 illustrates a temporal change in the pressure, which can be used as one operation parameter. Note that operation parameters included in physical indicators may vary depending on the type of sterilization processing apparatus 13. For example, the temperature or the like may also be included in the operation parameters. In the present embodiment, the sterilization processing apparatus 13 transmits data of a physical indicator to the management apparatus 14 via the network. The management apparatus 14 stores the received data of the physical indicator as a data file, and records the storage location (including the file name) of the data file, in a sterilization process history table.

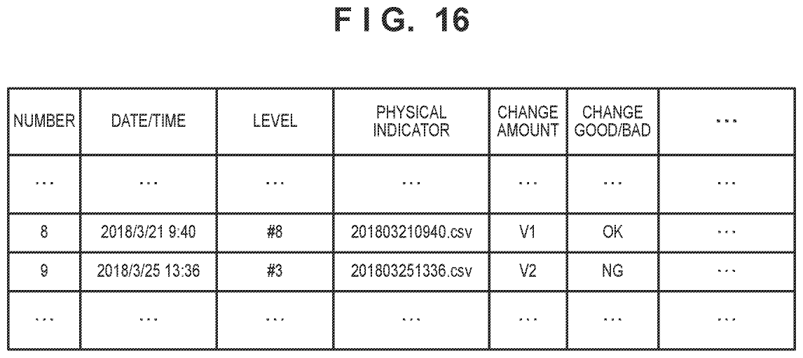

FIG. 16 illustrates the sterilization process history table according to the present embodiment. The history table includes a “Physical indicator” field for recording the storage location of the data file of the physical indicator. The history table also includes a “Change amount” field and a “Change good/bad” field, which will be described later. Note that the history table may also include the fields described in the first to third embodiments. In the present embodiment, a level shown in the level field is determined based on a CI that is packaged in a PCD. That is, each record in the history table of the present embodiment is a record of one sterilization process that is performed by the sterilization processing apparatus 13. However, a configuration is also possible in which each record is a record of the sterilization process that is performed on one set, as is the case with the first embodiment and the like, In this case, the same information is recorded in the physical indicator fields, the change amount fields, and the change good/bad fields of records that respectively correspond to a plurality of sets on which the sterilization process is simultaneously performed by the sterilization processing apparatus 13.

The management apparatus 14 holds a standard waveform of the physical indicator. The management apparatus 14 compares a waveform that is indicated by the received data file of the physical indicator of the sterilization process with the standard waveform, and determines a change amount from the standard waveform based on a predetermined standard. Then, the management apparatus 14 records the determined change amount in the change amount field. Note that a configuration is also possible in which a change level according to the change amount is recorded instead of the change amount itself. Specifically, a correspondence relationship between the range of the change amount and the change level is determined in advance and is set in the management apparatus 14. The management apparatus 14 records a change level that corresponds to the determined change amount, in the change amount field. For example, the change level can be set to decrease as the change amount increases. Alternatively, the change level can be set to increase as the change amount increases. in the following description, the change level decreases as the change amount increases. Further, in the following description, the change level includes the change amount itself, unless otherwise stated. If the change level is lower than a threshold value (i.e., bad), the management apparatus 14 records “NG” in the change good/bad field. in contrast, if the change level is equal to or higher than the threshold value (i.e., good), the management apparatus 14 records “OK” in the change good/bad field. In a case in which the change amount is used, “OK” is recorded if the change amount is equal to or smaller than a threshold value, and “NG” is recorded if the change amount is larger than the threshold value. Further, if “NG” is recorded in the change good/bad field, the management apparatus 14 can issue a warning to a user. Note that the warning can he issued to the user by displaying a message on a display of the management apparatus 14 or a display of a personal computer (PC, not shown) that is connected to the network, for example. Alternatively, the warning can be issued by outputting a warning sound from a speaker of the management apparatus 14 or a PC (not shown) that is connected to the network. Alternatively, the warning can be issued by transmitting an email to a predetermined user.

Next, the predetermined standard used for calculating the change amount will be described. For example, if entire waveforms are compared, a square error can be used as the change amount. That is, an integrated value of squares of amplitude differences from the standard waveform can be used as the change amount. Alternatively, an integrated value of amplitude differences from the standard waveform can be used as the change amount. If only a peak value of the waveform is important in the sterilization process, a difference between a peak value of the standard waveform and a peak value of the received physical indicator of the sterilization process can be used as the change amount. For example, if the physical indicator illustrated in FIG. 15 is received, a difference between a peak value P1 and a peak value of the standard waveform can be used as the change amount. If a period of duration of the peak value is also important in the sterilization process, as well as the peak value, it is also possible to use, as the change amount, a difference between a period of duration of the peak value of the standard waveform and a period of duration of the peak value of the received physical indicator of the sterilization process. For example, if the physical indicator illustrated in FIG. 15 is received, a difference between a period T2 of duration of the peak value P1 and a period of duration of the peak value of the standard waveform can also be used as the change amount. If different change amounts are calculated, a change level is determined for each of the calculated change amounts. Each change level is compared with a corresponding threshold value. If all change levels are equal to or higher than corresponding threshold values, “OK” is recorded in the change good/bad field, and if one or more change levels are lower than corresponding threshold values, “NG” is recorded in the change good/bad field. Further, if a period (T1 in FIG. 15) passed before the peak value is reached is also important in the sterilization process, a change amount that is based on this period is also taken into consideration. The predetermined standard is set in accordance with any other element that is important in the sterilization process. Although FIG. 15 illustrates the pressure, if the temperature or the like is also output as a physical indicator, the change amount and the change level are determined in the same manner as for the temperature as well. If all change levels are equal to or higher than threshold values, “OK” is recorded, otherwise “NG” is recorded.

Next, the standard waveform held by the management apparatus 14 will be described. A waveform that is recommended by the manufacturer of the sterilization processing apparatus can be used as the standard waveform, for example. Alternatively, the management apparatus 14 can determine the standard waveform based on information stored in a history table. For example, if Levels #8 and #9 are OK levels, the management apparatus 14 acquires data files that are indicated in the physical indicator fields of records for which #8 and #9 are recorded in the level fields. Then, the management apparatus 14 determines the standard waveform by averaging waveforms that are respectively indicated by these data files. Note that a configuration is also possible in which the standard waveform is determined only based on data files that are indicated in records for which Level #9 is recorded, even in a case in which Levels #8 and #9 are OK levels. That is, waveforms of the physical indicator that are used for determining the standard waveform are only required to be waveforms that are obtained in sterilization processes for which the achievement degree is at least a predetermined level (predetermined value). Note that the predetermined level is equal to or higher than a level for which a determination of “OK” is made. If a large sterilization processing apparatus 13 is used, the sterilization process can be simultaneously performed on a plurality of sets. In this case, the standard waveform is determined using waveforms that are indicated by physical indicators of records in which all sets simultaneously subjected to the sterilization process achieved the predetermined level or higher levels. Note that records that are used for determining the standard waveform can also be limited to past records within a predetermined period or a predetermined number of past records.

In the present embodiment, waveforms that indicate temporal changes in an operation parameter are recorded in the history table. However, if change amounts are determined using only a peak value, a period of duration of the peak value, and a period passed before the peak value is reached, a configuration is also possible in which only these values are recorded, rather than waveforms. In this case, the change amounts are calculated using standard values, rather than the standard waveform.

Also, the management apparatus 14 can present graphs that are similar to those described in the first to third embodiments, based on the history table. Specifically, information for determining transition of the quality of the sterilization process can be generated using change amounts or change levels in the change amount fields as values that are recorded in the level fields in the first to third embodiments. Alternatively, information for determining transition of the quality of the sterilization process can be generated using values in the change good/bad fields as values that are recorded in the good/bad fields in the first to third embodiments. Note that providing the change good/bad field corresponds to setting two levels as change levels. Values to be recorded in the average field, the standard deviation field, and the standard deviation exceeding ratio field, which are described in the first to third embodiments, are each calculated using values in the change amount fields. A value to be recorded in the ratio field, which is described in the first to third embodiments, is calculated using values in the change good/bad fields.

In the present embodiment, the sterilization processing apparatus 13 transmits a data file to the management apparatus 14. However, some sterilization processing apparatuses 13 do not have a communication function. If a sterilization processing apparatus 13 does not have a communication function and outputs a data file to a portable memory, such as a USB memory, for example, the operator can store the data file in the management apparatus 14 using the portable memory. There is also a sterilization processing apparatus 13 that outputs values of an operation parameter or a graph of the operation parameter onto paper by using a printer that is included in the sterilization processing apparatus 13, rather than outputting data, for example. In such a case, the operator can use any reading apparatus, such as an image scanner or a camera, to read the output paper, make a determination on the read values or graph of the operation parameter, and store a determined value or graph as a data file in the management apparatus 14. Then, the operator adds information for identifying the data file in the physical indicator field of the history table.

Function Blocks of Management Apparatus 14

The management apparatus 14 is a general-purpose computer including one or more processors, a volatile memory, and a non-volatile memory, and carries out the above-described processes by executing programs using the one or more processors. FIG, 17 is a function block diagram illustrating functions of the management apparatus 14 realized by executing the programs. A communication unit 144 communicates with the determination apparatus 1 and the sterilization processing apparatus 13 via the network. The communication unit 144 is also capable of communicating with a PC (not shown) that, is connected to the network. A holding unit 141 holds history tables and the standard waveform. Upon receiving a determination result from the determination apparatus 1, which is an external apparatus, a record processing unit 143 adds a record to a history table and records information in each field of the added record. Upon receiving data of a physical indicator from the sterilization processing apparatus 13, the record processing unit 143 stores the data in the holding unit 141 and records information for identifying the data file of the data in the physical indicator field of the relevant record. Further, upon receiving data of the physical indicator from the sterilization processing apparatus 13, the record processing unit 143 compares a waveform indicated by the data with the standard waveform and records a change amount or a change level in the change amount field. Further, the record processing unit 143 records the result (OK or NG) of the comparison between the change amount or the change level and a threshold value, in the change good/bad field.

A generating unit 142 generates information for determining transition of the quality of the sterilization process based on records in the history table, as described in the first to fourth embodiments, and displays graphs, such as those described in the embodiments, on a display based on the generated information. Note that a configuration is also possible in which the generated information is output to a printer or is stored in a memory instead of or in addition to being displayed on the display. In the above-described embodiments, information for determining transition of the quality of the sterilization process is generated based on all records in a history table, but a configuration is also possible in which information for determining transition of the quality of the sterilization process is generated based on records for which the date/time shown in the date/time field is within a predetermined period. In this case, the predetermined period is set by the user, for example.

Also, the generating unit 142 determines the standard waveform (standard value) based on a history table as described in the fourth embodiment. Also, if the result of the comparison is determined as NG by the record processing unit 143, a notification unit 145 issues a warning to the user. That is, the notification unit 145 carries out a process for notifying the user that the change amount of the physical indicator relative to a standard has exceeded a threshold value.

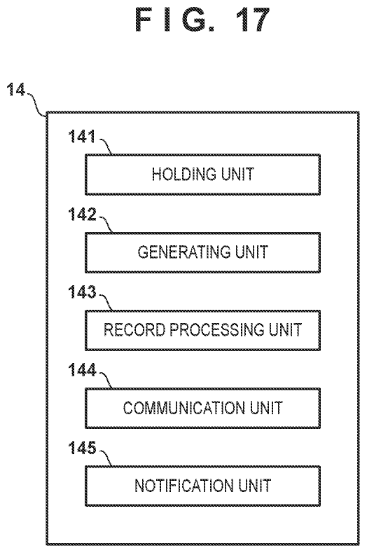

Note that the present invention is not limited to a form in which the function blocks illustrated in FIG. 17 are realized by a general-purpose computer. Some or all of the function blocks illustrated in FIG. 17 can be realized by any desired dedicated hardware. In the above-described embodiments, the determination apparatus 1 determines the level of the sterilization process and whether the sterilization process is good or bad, but a configuration is also possible in which the management apparatus 14 determines the level of the sterilization process and whether the sterilization process is good or bad. In this case, the determination apparatus 1 functions as a color measuring apparatus that measures the color of the discoloration area 21 of the CI 2 and transmits the result of the measurement (color measurement result) of the discoloration area 21 of the CI 2 to the management apparatus 14. The management apparatus 14 is provided with a determination unit that determines the level of the sterilization process and whether the sterilization process is good or bad based on the measurement result, and records determination results in relevant fields of the record, in the above-described embodiments, the use of a plurality of determination apparatuses is assumed, and the management apparatus 14 that manages history tables is configured as an apparatus that is different from the determination apparatuses 1. However, if only one determination apparatus 1 is used, the management apparatus 14 and the determination apparatus 1 can be configured as one apparatus.

Further, a configuration is also possible in which the functions of the management apparatus 14 illustrated in FIG. 17 are realized by a plurality of computers that can communicate with each other via a network, rather than being realized by one computer.

In the above-described embodiments, records of achievement degrees of the sterilization process and the washing process are managed. However, the present invention can be applied to records of any process that is carried out using test paper that has a discoloration area that changes color in accordance with the achievement degree of the process. The present invention can also be applied to any process that is carried out using a processing apparatus that is capable of outputting temporal changes in physical parameters during the process.

Regarding the CI

As illustrated in FIG. 2, in the above embodiments, the CI 2 has a rectangular discoloration area 21, which is of a color different from the backing. The color of the discoloration area 21 changes in accordance with the achievement degree of the sterilization process. The “color” of the discoloration area 21 also includes a transparent state. That is, even if the discoloration area 21 is initially transparent, and a color appears in accordance with the achievement degree of the sterilization process, that situation is included in the concept of the “discoloration” or “color change” according to the present invention. Likewise, even if the discoloration area 21 is not transparent initially, but the discoloration area 21 becomes transparent in accordance with the achievement degree of the sterilization process, that situation is included in the concept of the “discoloration” or “color change” according to the present invention. The base of CI 2 may be transparent as well.

Additionally, the color of the discoloration area 21 may initially be the same as the color of the base, for example. Furthermore, the entire discoloration area 21 need not change color uniformly in accordance with the achievement degree of the sterilization process. For example, even if the entire discoloration area 21 initially has the same color as the base, but a lengthwise part thereof changes to a color different from that of the base in accordance with the achievement degree of the sterilization process, that situation is included in the concept of the “discoloration” or “color change” according to the present invention. Note that the discoloration area 21 may have a part that has a color different from the base initially, with the part having a color different from the base growing longer in accordance with the achievement degree of the sterilization process. Conversely, the part having a color different from the base may grow shorter in accordance with the achievement degree of the sterilization process. With such a CI 2, the achievement degree of the sterilization process is determined from the length of the part in which the color of the discoloration area 21 has changed, or the length of the part in which the color has not changed.

### Other Embodiments

Embodiments of the present invention can also be realized by a computer of a system or apparatus that reads out and executes computer executable instructions (e.g., one or more programs) recorded on a storage medium (which may also he referred to more fully as a ‘non-transitory computer-readable storage medium’) to perform the functions of one or more of the above-described embodiments and/or that includes one or more circuits (e.g., application specific integrated circuit (ASIC)) for performing the functions of one or more of the above-described embodiments, and by a method performed by the computer of the system or apparatus by, for example, reading out and executing the computer executable instructions from the storage medium to perform the functions of one or more of the above-described embodiments and/or controlling the one or more circuits to perform the functions of one or more of the above-described embodiments. The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2018-222690, filed on Nov. 28, 2018, and Japanese Patent Application No. 2019-131400, filed on Jul. 16, 2019 which are hereby incorporated by reference herein in their entirety.

1. A management apparatus that manages records of a predetermined process carried out using test paper, the test paper having a discoloration area that changes color in accordance with an achievement degree of the predetermined process, the apparatus comprising:
one or more processors; and
one or more memories including a program,
wherein the program, when executed by the one or more processors, causes the one or more processors to perform operations including:
upon the predetermined process being performed, recording, in a record, information that indicates a date/time at which the predetermined process was carried out and a determination result of the predetermined process determined based on the color of the discoloration area of the test paper; and
generating information for determining transition of quality of the predetermined process based on the information recorded in the records.

2. The management apparatus according to claim 1,
wherein the operations further include generating, as the information for determining transition of the quality of the predetermined process, information that indicates transition of a moving average of achievement degrees that are respectively recorded in records arranged in chronological order.

3. The management apparatus according to claim 1,
wherein the operations further include generating, as the information for determining transition of the quality of the predetermined process, information that indicates transition of a standard deviation of achievement degrees that are recorded in a predetermined number of successive records arranged in chronological order.

4. The management apparatus according to claim 1,
wherein the operations further include generating, as the information for determining transition of the quality of the predetermined process, a relational expression between the date/time and the achievement degree, the relational expression being determined based on the date/time and achievement degrees that are recorded in the records.

5. The management apparatus according to claim 4,
wherein the operations further include:
calculating a standard deviation of the achievement degree based on the achievement degrees that are recorded in the records used for determining the relational expression, and
generating, as the information for determining transition of the quality of the predetermined process, information that indicates a relationship between the date/time and a probability of the achievement degree of the predetermined process being achieved, the relationship being determined based on the relational expression and the standard deviation.

6. The management apparatus according to claim 1,
wherein the operations further include:
calculating, for each record, a moving average of achievement degrees that are respectively recorded in records arranged in chronological order and a standard deviation of achievement degrees that are recorded in a predetermined number of successive records arranged in chronological order,
determining, for each record, a comparison result that indicates whether a difference between the achievement degree recorded in the record and the moving average calculated for the record is larger than a threshold value that is determined based on the standard deviation calculated for the record,
calculating, for each record, a ratio at which the difference is larger than the threshold value or a ratio at which the difference is equal to or smaller than the threshold value, based on the comparison results determined for a predetermined number of successive records arranged in chronological order, and
generating, as the information for determining transition of the quality of the predetermined process, a relational expression between the date/time and the ratio, the relational expression being determined based on the date/time recorded in each record and the ratio calculated for each record.

7. The management apparatus according to claim 1,
wherein the determination result indicates whether the predetermined process is good or bad, and
the operations further include:
calculating, for each record, a ratio of good or bad that is recorded in a predetermined number of successive records arranged in chronological order, and
generating, as the information for determining transition of the quality of the predetermined process, a relational expression between the date/time and the ratio, the relational expression being determined based on the date/time recorded in each record and the ratio calculated for each record.

8. The management apparatus according to claim 4,
wherein the operations further include predicting when the quality of the predetermined process is to reach a predetermined level based on the relational expression.

9. The management apparatus according to claim 1,
wherein related information that indicates an object or person relating to the predetermined process is recorded in each of the records, and
the operations further include generating the information for determining transition of the quality of the predetermined process with respect to the object or person indicated by the related information, based on records in which the same related information is recorded.

10. The management apparatus according to claim 1,
wherein the operations further include:
adding the record to a history table upon receiving a measurement result of the color of the discoloration area of the test paper from an external apparatus,
determining a result of the predetermined process based on the measurement result, and
recording the determined result, as the determination result, in the added record.

11. The management apparatus according to claim 1,
wherein the operations further include:
adding the record to a history table upon receiving the determination result from an external apparatus, and
recording the received determination result in the added record.

12. The management apparatus according to claim 10,
wherein the date/time is received together with the measurement result or the determination result from the external apparatus.

13. The management apparatus according to claim 10,
wherein the date/time is held by the management apparatus when the measurement result or the determination result is received from the external apparatus.

14. The management apparatus according to claim 1,
wherein the predetermined process is a sterilization process or a washing process.

15. A management apparatus that manages records of a predetermined process carried out by a processing apparatus, the management apparatus comprising:
one or more processors; and
one or more memories including a program,
wherein the program, when executed by the one or more processors, causes the one or more processors to perform operations including:
upon the predetermined process being performed, recording, in a record, information that indicates a date/time at which the predetermined process was carried out, an operation parameter of the processing apparatus that is output by the processing apparatus, and a change level of the operation parameter relative to a standard value; and
generating information for determining transition of quality of the predetermined process based on the information recorded in the records.

16. The management apparatus according to claim 15,
wherein the operations further include generating, as the information for determining transition of the quality of the predetermined process, information that indicates transition of a moving average of the change levels that are respectively recorded in records arranged in chronological order.

17. The management apparatus according to claim 15,
wherein the operations further include generating, as the information for determining transition of the quality of the predetermined process, information that indicates transition of a standard deviation of the change levels that are recorded in a predetermined number of successive records arranged in chronological order.

18. The management apparatus according to claim 15,
wherein the operations further include generating, as the information for determining transition of the quality of the predetermined process, a relational expression between the date/time and the change level, the relational expression being determined based on the date/time and change levels that are recorded in the records.

19. The management apparatus according to claim 18,
wherein the operations further include:
calculating a standard deviation of the change level based on the change levels that are recorded in the records used for determining the relational expression, and
generating, as the information for determining transition of the quality of the predetermined process, information that indicates a relationship between the date/time and a probability of the change level being achieved, the relationship being determined based on the relational expression and the standard deviation.

20. The management apparatus according to claim 15,
wherein the operations further include:
calculating, for each record, a moving average of the change levels that are respectively recorded in records arranged in chronological order and a standard deviation of the change levels that are recorded in a predetermined number of successive records arranged in chronological order,
determining, for each record, a comparison result that indicates whether a difference between the change level recorded in the record and the moving average calculated for the record is larger than a first threshold value that is determined based on the standard deviation calculated for the record,
calculating, for each record, a ratio at which the difference is larger than the first threshold value or a ratio at which the difference is equal to or smaller than the first threshold value, based on the comparison results determined for a predetermined number of successive records arranged in chronological order, and
generating, as the information for determining transition of the quality of the predetermined process, a relational expression between the date/time and the ratio, the relational expression being determined based on the date/time recorded in each record and the ratio calculated for each record.

21. The management apparatus according to claim 15,
wherein the change level indicates good or bad, and
the operations further include:
calculating, for each record, a ratio of good or bad that is recorded in a predetermined number of successive records arranged in chronological order, and
generating, as the information for determining transition of the quality of the predetermined process, a relational expression between the date/time and the ratio, the relational expression being determined based on the date/time recorded in each record and the ratio calculated for each record.

22. The management apparatus according to claim 18,
wherein the operations further include predicting when the quality of the predetermined process is to reach a predetermined level based on the relational expression.

23. The management apparatus according to claim 15,
wherein related information that indicates an object or person relating to the predetermined process is recorded in each of the records, and
the operations further include generating the information for determining transition of the quality of the predetermined process with respect to the object or person indicated by the related information, based on records in which the same related information is recorded.

24. The management apparatus according to claim 15,
wherein the operations further include notifying a user that the change level is larger than a second threshold value, when the change level is larger than the second threshold value.

25. The management apparatus according to claim 15
wherein the predetermined process is carried out using test paper that has a discoloration area that changes color in accordance with an achievement degree of the predetermined process,
the achievement degree of the predetermined process that is determined based on the color of the discoloration area of the test paper is recorded in each of the records, and
the operations further include generating the standard value based on the operation parameters indicated by information that is recorded in records for which the recorded achievement degree is higher than a predetermined value.

26. The management apparatus according to claim 15,
wherein the operation parameter indicated by the information recorded in the records is at least one of a peak value of a physical amount relating to operation of the processing apparatus, a period of duration of the peak value, and a period passed before the peak value is reached.

27. The management apparatus according to claim 15,
wherein the operation parameter indicated by the information recorded in the records is a waveform that indicates a temporal change in a physical amount relating to operation of the processing apparatus.

28. The management apparatus according to claim 15,
wherein the change level is a change amount of the operation parameter relative to a standard value.

29. A non-transitory computer readable storage medium including a program that, when executed by one or more processors in an apparatus including the one or more processors, causes the apparatus to function as a management apparatus that manages records of a predetermined process carried out using test paper, the test paper having a discoloration area that changes color in accordance with an achievement degree of the predetermined process and to perform operations including:
upon the predetermined process being performed, recording, in a record, information that indicates a date/time at which the predetermined process was carried out and a determination result of the predetermined process determined based on the color of the discoloration area of the test paper; and
generating information for determining transition of quality of the predetermined process based on the information recorded in the records.

30. A management system that manages records of a predetermined process carried out using test paper, the test paper having a discoloration area that changes color in accordance with an achievement degree of the predetermined process, the system comprising:
a holding unit configured to hold, for each predetermined process, a record in which information that indicates a date/time at which the predetermined process was carried out and a determination result of the predetermined process determined based on the color of the discoloration area of the test paper is recorded; and
a generating unit configured to generate information for determining transition of quality of the predetermined process based on the information recorded in the records.

31. The management system according to claim 30,
wherein the generating unit generates, as the information for determining transition of the quality of the predetermined process, information that indicates transition of a moving average of achievement degrees that are respectively recorded in records arranged in chronological order.

32. The management system according to claim 30,
wherein the generating unit generates, as the information for determining transition of the quality of the predetermined process, a relational expression between the date/time and the achievement degree, the relational expression being determined based on the date/time and achievement degrees that are recorded in the records.

33. The management system according to claim 30,
wherein the generating unit calculates, for each record, a moving average of achievement degrees that are respectively recorded in records arranged in chronological order and a standard deviation of achievement degrees that are recorded in a predetermined number of successive records arranged in chronological order, determines, for each record, a comparison result that indicates whether a difference between the achievement degree recorded in the record and the moving average calculated for the record is larger than a threshold value that is determined based on the standard deviation calculated for the record, calculates, for each record, a ratio at which the difference is larger than the threshold value or a ratio at which the difference is equal to or smaller than the threshold value, based on the comparison results determined for a predetermined number of successive records arranged in chronological order, and generates, as the information for determining transition of the quality of the predetermined process, a relational expression between the date/time and the ratio, the relational expression being determined based on the date/time recorded in each record and the ratio calculated for each record.

34. The management system according to claim 30,
wherein the determination result indicates whether the predetermined process is good or bad, and
the generating unit calculates, for each record, a ratio of good or bad that is recorded in a predetermined number of successive records arranged in chronological order, and generates, as the information for determining transition of the quality of the predetermined process, a relational expression between the date/time and the ratio, the relational expression being determined based on the date/time recorded in each record and the ratio calculated for each record.

35. The management system according to claim 30,
wherein related information that indicates an object or person relating to the predetermined process is recorded in each of the records, and
the generating unit generates the information for determining transition of the quality of the predetermined process with respect to the object or person indicated by the related information, based on records in which the same related information is recorded.

36. The management system according to claim 30, further comprising:
a determination unit configured to determine a result of the predetermined process based on a measurement result of the color of the discoloration area of the test paper; and
a recording unit configured, when the determination is made by the determination unit, add the record to a history table and record the result determined by the determination unit in the added record.

37. The management system according to claim 36, further comprising a measuring unit configured to measure the color of the discoloration area of the test paper.

38. A method for generating information for determining transition of the quality of a predetermined process carried out using test paper, the test paper having a discoloration area that changes color in accordance with an achievement degree of the predetermined process, the method comprising:
upon the predetermined process being performed, recording, in a record, information that indicates a date/time at which the predetermined process was carried out and a determination result of the predetermined process determined based on the color of the discoloration area of the test paper; and
generating information for determining transition of quality of the predetermined process based on the information recorded in the record.
